(19) United States
(12) Patent Application Publication
(10) Pub. No.: US 2024/0425399 A1
(43) Pub. Date: Dec. 26, 2024

(54) MANIPULATION OF ALGAL MICROBIOME COMPOSITION WITH SILICON-RICH MATERIALS FOR ENHANCED PHOSPHORUS REMOVAL FROM WASTEWATER

(71) Applicant: Alliance for Sustainable Energy, LLC, Golden, CO (US)

(72) Inventors: Jianping YU, Golden, CO (US); Eric Ryan SCHAEDIG, Denver, CO (US)

(21) Appl. No.: 18/752,425

(22) Filed: Jun. 24, 2024

Related U.S. Application Data
(60) Provisional application No. 63/523,014, filed on Jun. 23, 2023.

(51) Int. Cl. C02F 3/32; C02F 101/10; C12N 1/12; C12R 1/89

(52) U.S. Cl. CPC C02F 3/322; C12N 1/125; C02F 2101/105; C12R 2001/89

(57) ABSTRACT

Excess phosphorus (P) in wastewater effluent poses a serious threat to aquatic ecosystems and can spur harmful algal blooms. Revolving algal biofilm (RAB) systems are an emerging technology to recover P from wastewater before discharge into aquatic ecosystems. In RAB systems, a community of microalgae take up and store wastewater P as polyphosphate as they grow in a partially submerged revolving biofilm, which may then be harvested and dried for use as fertilizer in lieu of mined phosphate rock. Disclosed herein are methods for isolating and characterizing microalgae strains from active RAB systems. Strains were identified by microscopy and 16S/18S ribosomal DNA sequencing, cryopreserved, and screened for elevated P content (as polyphosphate). Seven isolated strains possessed at least 50% more polyphosphate by cell dry weight than a microalgae consortium from a RAB system, with the top strain accumulating nearly threefold more polyphosphate. Isolated P-hyperaccumulating microalgae have broad applications in resource recovery from various waste streams, including improving P removal from wastewater.

### CROSS-REFERENCE TO RELATED APPLICATIONS

This application is a continuation in part of and claims priority under 35 U.S.C. § 119 to U.S. Provisional Patent Application No. 63/523,014 filed on 23 Jun. 2023, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Wastewater treatment facilities face increasingly strict phosphorus discharge limits from government agencies, and traditional nutrient removal methods are either insufficient, too complicated, or too expensive or energy intensive. Algae-based nutrient removal is promising and is being deployed worldwide.

Microalgae are an emerging tool for phosphorus (P) recovery and reuse technology. Phosphorus is a vital nutrient for all life on Earth, playing essential roles in biomolecule synthesis, cellular signaling, and energy storage. Anthropogenic manipulation of the P cycle for agriculture and other applications has led to global imbalances in P distribution. These imbalances are particularly pronounced in aquatic ecosystems, which receive excess P via several pathways including runoff from agricultural land and wastewater effluent discharge into waterways. These excess P inputs lead to eutrophication of aquatic ecosystems that may spur harmful algal blooms. In response to this persistent and growing issue, United States federal and state agencies overseeing water quality have tightened P emission limits in recent years, and these limits are expected to become increasingly strict. Conversely, human processes such as agriculture and manufacturing currently rely heavily on finite geological stores of high-grade phosphate rock. New technologies are critically needed to not only limit P emissions to the environment but also to reclaim P from waste streams to alleviate global reliance on finite phosphate rock resources.

Wastewater is an attractive target for the development of such nutrient reclamation technologies because of its nutrient-rich composition. A typical conventional wastewater treatment plant can remove only 10% of total P from raw municipal influent through solids settling and 30% of total P through biological metabolism in conventional activated sludge. However, more recent data from conventional activated sludge treatment plants operated by the Metropolitan Water Reclamation District of Greater Chicago (MWRD Chicago) shows total P removal by activated sludge to range between 42% to 57%. These removal rates are not sufficient to meet discharge limits, and the remaining P is often removed from wastewater as needed by precipitation with trivalent metal cations due to the high efficiency and low technical demand of the method. However, these phosphate precipitates are difficult to separate and reuse as bioavailable P, incurring a waste disposal burden on wastewater treatment plants and limiting the sustainability of this practice. Phosphorus precipitation as magnesium ammonium phosphate (struvite) may also be used to efficiently remove P from water, and the captured P may be reused. However, issues with this method, including high operational costs and energy consumption, have slowed the widespread adoption of struvite precipitation. Enhanced biological phosphorus removal may also be used to remove P from wastewater, and MWRD Chicago facilities operating the technology have demonstrated total P removal rates between 84% to 92%. However, this method is operationally complex, energy-intensive, prone to fluctuations, and often restricted to large wastewater treatment facilities.

Microalgae, or microscopic algae, are an emerging and powerful tool to reclaim P from wastewater. Microalgae are capable of growing in wastewater and take up P from the wastewater as a critical nutrient for biomolecule synthesis. However, microalgae often take up P at levels surpassing their nutritional needs (commonly referred to as “luxury uptake”). In the luxury uptake process, microalgae store excess P intracellularly as polyphosphate (polyP), a high molecular weight polymer of inorganic phosphate. This process has made microalgae a topic of interest in biological wastewater P recovery efforts.

Microalgae have long been used to treat wastewater and remove nutrients and organic pollutants in open pond systems. More recently, Revolving Algal Biofilm (RAB) systems have been developed to improve the efficiency of nutrient removal from wastewater by optimizing nutritional and light conditions to increase microalgal growth. Microalgae present in the wastewater colonize the RAB system to form an attached biofilm on a vertical semi-submerged revolving belt. Exposure of the biofilm to the atmosphere allows gas exchange and light penetration into the biofilm, while intermittent submersion in the wastewater allows for adsorption and cellular uptake of P and other pollutants by the microalgae as they grow. The nutrient-rich microalgal biomass may then be harvested by scraping and used as fertilizer or as feedstock to produce biofuel or other bioproducts. The RAB system design has been optimized in recent years to improve rates of nutrient removal and biomass production through an engineering approach. However, RAB systems have not yet been optimized from a biological perspective. Manipulating the microalgae species that colonize and grow in these systems may lead to improvements in nutrient recovery, biomass production, and other performance parameters.

Luxury uptake is reported to be dynamic and varies across different species of microalgae, suggesting that RAB system P recovery may be biologically enhanced by introducing strains of microalgae capable of hyperaccumulating P. Many studies have demonstrated elevated P levels attributable to luxury uptake in both environmental microalgal biomass and individual strains of green algae and cyanobacteria cultivated in wastewater. For example, it has been previously reported that transient polyP levels of up to 24.5% cell dry weight (CDW) in the green alga Chlorella protothecoides when cultivated in primary municipal wastewater. However, RAB systems cultivate microalgae in a unique revolving aerial biofilm system that may be inhospitable to many previously identified P-hyperaccumulating strains. Thus, the isolation and characterization of new P-hyperaccumulating microalgae strains native to the RAB system is prudent to ensure the isolates are capable of recolonization, a necessary step in improving the P removal efficiency of the system.

### SUMMARY

In an aspect, disclosed herein is a method for enriching a microbial population capable of sequestering phosphorus and nitrogen comprising the addition of silicon rich materials to a solution wherein the microbial population is contacted with the solution. In an embodiment, the silicon rich material is sand. In an embodiment, the microbial population is cyclically exposed to the solution and further exposed to atmosphere. In an embodiment, the atmosphere comprises carbon dioxide.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts an embodiment of the systems and method disclosed herein.

FIG. 2 depicts light microscopy images of RAB biofilm samples. (A) Biofilm sampled in April 2021 from a demonstration-scale RAB treating municipal final clarifier effluent in Creston, Iowa. (B) Biofilm sampled in December 2020 from a pilot-scale RAB treating industrial (meat processing) anaerobic digester effluent in Sioux City, Iowa. (C) Biofilm sampled in April 2021 from a demonstration-scale RAB treating municipal tertiary effluent in Chicago, Illinois. (D) Biofilm sampled in December 2020 from a demonstration-scale RAB treating municipal secondary effluent in Slater, Iowa. (E) Biofilm sampled in February 2021 from a pilot-scale RAB treating municipal intermediate clarifier effluent in Creston, Iowa.

FIG. 3 depicts light microscopy images of isolated RAB microalgae. Strains were identified by 16S/18S ribosomal DNA sequencing. (A) Green alga Desmodesmus sp. TCF-3g isolated from Chicago, Illinois. (B) Green alga Caespitella pascheri TCF-75g isolated from Creston, Iowa. (C) Cyanobacterium Pseudanabaena sp. TCF-9c isolated from Creston, Iowa. (D) Cyanobacterium Nostoc edaphicum TCF-1c isolated from Chicago, Illinois. (E) Diatom Craticula molestiformis TCF-1d isolated from Creston, Iowa. (F) Diatom Nitzschia palea TCF-4d isolated from Creston, Iowa.

FIG. 4 depicts Polyphosphate content of isolated RAB microalgae. Unique microalgae were screened for polyphosphate content, along with a RAB microalgae consortium from a RAB system treating municipal wastewater in Slater, Iowa. Data depicts measured polyphosphate as a percentage of cell dry weight (CDW). Light microscopy images of the top P-hyperaccumulating morphologically unique strains and the RAB microalgae consortium are shown. Error bars reflect the standard deviation of triplicate fluorescence measurements of extracted polyphosphate from a single strain culture or consortium sample.

FIG. 5 depicts biomass productivity, phosphorus content, and accumulated P of microalgae grown in bench-scale RAB systems containing BBM medium. Two isolated microalgae were cultivated over three weeks in bench-scale RAB systems alongside a RAB microalgae consortium from a RAB system treating municipal wastewater in Slater, Iowa. (A) Four bench-scale RAB systems used to cultivate (from left to right) Chlorellaceae sp. TCF-17g, Chlamydomonas pulvinata TCF-48g, both strains together, and the RAB microalgae consortium (B) Total dry biomass productivity of the four RAB systems over the course of three weeks. (C) Phosphorus content of biomass harvested from each RAB system across the first harvest (7 days) and subsequent pooled harvests (14 and 21 days). (D) Total accumulated phosphorus by each RAB system over three weeks of growth (calculated from biomass productivity & P content). The dashed blue line represents the total amount of phosphorus added to each system throughout the experiment (424 mg).

FIGS. 6A, 6B, 6C, 6D. Microscopy images of the bench-scale RAB biofilms seven days after inoculation. Objective magnification at 100×. Arrows point to cells matching the morphology of the inoculated strain(s). (A) Biofilm inoculated with Chlamydomonas pulvinata TCF-48g. (B) Biofilm inoculated with Chlorellaceae sp. TCF-17g. (C) Biofilm inoculated with both Chlamydomonas pulvinata TCF-48g and Chlorellaceae sp. TCF-17g. (D) Biofilm inoculated with the RAB microalgae consortium from Slater, Iowa

### DETAILED DESCRIPTION

In an embodiment, disclosed herein are methods to increase phosphorus removal rates in algae-based wastewater treatment systems, thus enabling the wider deployment of such systems and help wastewater treatment facilities meet phosphorus discharge limits while also reducing carbon footprint.

In an embodiment, disclosed herein are methods useful for the isolation of algae that hyper-accumulate P. Using methods disclosed herein, a total of 101 microalgae strains from active RAB systems across the US Midwest, including 82 green algae, 9 diatoms, and 10 cyanobacteria were isolated and characterized. Strains were identified by microscopy and 16S/18S ribosomal DNA sequencing, cryopreserved, and screened for elevated P content (as polyphosphate). Seven isolated strains possessed at least 50% more polyphosphate by cell dry weight than a microalgae consortium from a RAB system, with the top strain accumulating nearly threefold more polyphosphate. These top P-hyperaccumulating strains include the green alga Chlamydomonas pulvinata TCF-48g and the diatoms Eolimna minima TCF-3d and Craticula molestiformis TCF-8d, possessing 11.4%, 12.7%, and 14.0% polyphosphate by cell dry weight, respectively. As a preliminary test of strain application for recovering P, Chlamydomonas pulvinata TCF-48g was reinoculated into a bench-scale RAB system containing BBM medium. The strain successfully recolonized the system and recovered twofold more P from the medium than a microalgae consortium from a RAB system treating municipal wastewater. These isolated P-hyperaccumulating microalgae may have broad applications in resource recovery from various waste streams, including improving P removal from wastewater.

In an embodiment, methods are disclosed herein to improve RAB system performance by building a culture collection of microalgae isolated from RAB systems treating wastewater across the United States Midwest with the aim of isolating P-hyperaccumulating microalgae. Isolates were characterized by microscopy, identified by 16S/18S ribosomal DNA (rDNA) sequencing, screened for elevated polyP content, and cryopreserved for future use. As a preliminary proof of principle test that isolated strains may be reintroduced to enhance system performance, two isolated strains were cultivated in bench-scale RAB systems containing artificial medium, and biomass productivity and P uptake were measured in comparison to a consortium of microalgae sampled from a RAB system treating municipal wastewater.

Excess phosphorus (P) in wastewater effluent poses a serious threat to aquatic ecosystems and can spur harmful algal blooms. Microalgae have been successfully used to treat wastewater and remove nutrients in open pond systems, tubular photobioreactor systems, and biofilm reactor systems. So far, the microalgae used in such systems are naturally formed consortia which are not optimized in terms of microbiome composition for enhanced phosphorus removal. We have recently discovered that among algal strains in the wastewater treatment microbiome, diatoms accumulate high levels of phosphorus relative to green algae and cyanobacteria. Diatoms require Si compounds to form cell walls and support their growth, but Si supply in wastewater can be limiting for diatom growth. We propose that addition of Si-rich compounds will enrich diatoms among the algal microbiome, leading to enhanced phosphorus removal rates in algae-based wastewater treatment systems. The Si-rich compounds include diatomaceous earth, sand powder, and other materials. Such compounds can be added to the treatment system, and their effect on the enrichment of diatoms can be easily monitored via microscopy, and their effect on phosphorus removal can be monitored by water chemistry analysis.

Currently, phosphorus in wastewater is most commonly removed by precipitation with trivalent metal cations due to its high efficacy and low technical demand. Metal salt precipitates of phosphorus are difficult to separate, however, preventing efficient reuse of the phosphorus. Alternatively, phosphorus can be removed biologically through processes like enhanced biological phosphorus removal (EBPR) in conventional activated sludge treatment systems. The EBPR process requires high levels of technical expertise and maintenance to achieve reliable phosphorus removal from wastewater influents, making it an unattractive option for smaller-scale wastewater treatment plants. In contrast, algal wastewater treatment systems recover reusable phosphorus and are suitable for both small- and large-scale applications. This technology would optimize the microbial ecology of these systems to improve performance. No current methods exist that manipulate algal microbiome composition to improve wastewater treatment performance.

Excess phosphorus (P) in wastewater effluent poses a serious threat to aquatic ecosystems and can spur harmful algal blooms. RAB systems are an emerging technology to recover P from wastewater before discharge into aquatic ecosystems. In RAB systems, a community of microalgae take up and store wastewater P as polyphosphate as they grow in a partially submerged revolving biofilm, which may then be harvested and dried for use as fertilizer in lieu of mined phosphate rock. We isolated and characterized a total of 101 microalgae strains from active RAB systems across the US Midwest, including 82 green algae, 9 diatoms, and 10 cyanobacteria. Strains were identified by microscopy and 16S/18S ribosomal DNA sequencing, cryopreserved, and screened for elevated P content (as polyphosphate). Seven isolated strains possessed at least 50% more polyphosphate by cell dry weight than a microalgae consortium from a RAB system, with the top strain accumulating nearly threefold more polyphosphate. These top P-hyperaccumulating strains include the green alga Chlamydomonas pulvinata TCF-48g and the diatoms Eolimna minima TCF-3d and Craticula molestiformis TCF-8d, possessing 11.4%, 12.7%, and 14.0% polyphosphate by cell dry weight, respectively. As a preliminary test of strain application for recovering P, Chlamydomonas pulvinata TCF-48g was reinoculated into a bench-scale RAB system containing Bold basal medium (BBM). The strain successfully recolonized the system and recovered twofold more P from the medium than a microalgae consortium from a RAB system treating municipal wastewater. These isolated P-hyperaccumulating microalgae may have broad applications in resource recovery from various waste streams, including improving P removal from wastewater.

### 2. Materials and Methods

### 2.1 Media Composition, Sample Collection, and Microalgae Isolation

A variety of media compositions were used to isolate and cultivate microalgae from RAB samples. These include bold basal medium supplemented with 100 μg L-1 cyanocobalamin (vitamin B12; Sigma), BG-11, and synthetic wastewater medium (GWT-SE) (Table 1). GWT-SE medium composition was modified to mimic the physicochemical properties and nutrient content of secondary municipal sewage effluent (Table 2). Liquid medium was autoclaved or filter-sterilized with a 0.22 μm polyethersulfone bottle-top filter (Corning). Solid medium was prepared by the addition of agar (BD, 1.5% w/v final concentration). Antibiotics, buffers, and other reagents were sterilized with a 0.22 μm polyethersulfone bottle-top filter or a 0.22 μm polyvinylidene fluoride syringe filter (NEST Scientific).

TABLE 1
Medium compositions used to isolate and
cultivate microalgae from RAB systems.

| | | | |
|---|---|---|---|
| | BBM | BG-11 | BBM |
| GWT-SE | (Nichols and | (Rippka et | (Nichols and |
| (Table S2) | Bold, 1965) | al., 1979) | Bold, 1965) |
| 1-10X | 1X only | 1X only | 1X only |

| | |
|---|---|
| 100 μg L⁻¹ cyanocobalamin (BBM only) | 100 μg L⁻¹ cyanocobalamin |
| 5 g L⁻¹ SiO₂* | 10 g L⁻¹ diatomaceous earth* |
| 100 μg mL⁻¹ to 1 mg mL⁻¹ sodium | 8.4 g L⁻¹ sodium bicarbonate^{‡} |
| ampicillin^{†} | |
| 1 μg mL⁻¹ to 5 μg mL⁻¹ carbendazim^{†} | |

*Included only for diatom isolates. Diatomaceous earth (Sigma) was added to liquid media, autoclaved at 121° C. for 60 min, and clarified with a 0.22 μm PES bottle-top filter.
^{†}Included only to axenize microalgal isolates.
^{‡}Included only for unicellular green algae and cyanobacteria. Dissolved in 1/10^{th} volume of prepared medium and pH adjusted to 7.5-7.6 with HCl before re-addition.

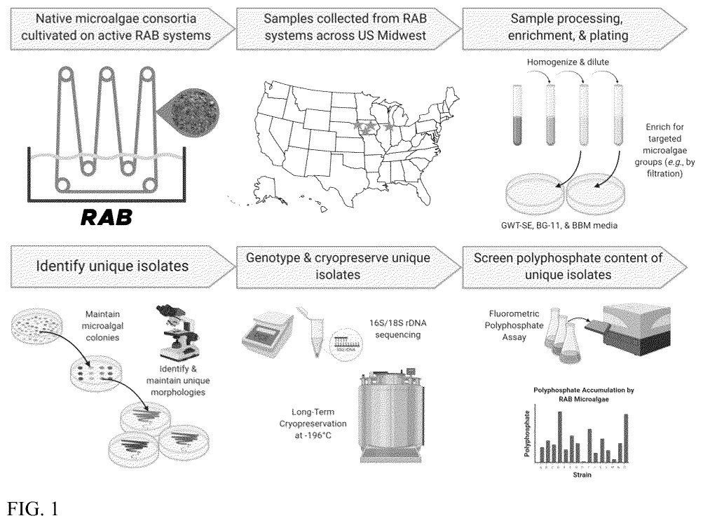

RAB samples were collected from active RAB systems across the United States Midwest in December 2020, February 2021, and April 2021. Collected samples were shipped overnight in 50 mL conical polypropylene tubes to the National Renewable Energy Laboratory in Golden, Colorado, USA for use in this study (FIG. 1). Upon receipt, RAB samples were stirred, and 1 to 5 mL of sample was transferred to 125-250 mL borosilicate Erlenmeyer flasks containing liquid GWT-SE medium and placed under white LED panel lights at 70-130 μE m⁻² s⁻¹ for long-term maintenance. All cultivation in this study was conducted at 26° C. RAB samples and flask cultures were imaged via brightfield microscopy with a Carl Zeiss Axio Scope.A1 microscope. Microscopic images were taken with an AxioCam MRc digital camera (Carl Zeiss) and AxioVision software (Carl Zeiss; v4.8.2).

TABLE 2
Chemical composition of GWT-SE synthetic secondary municipal wastewater effluent medium.

| | | | | | | NO₂⁻/ | |
|---|---|---|---|---|---|---|---|
| | | | Total | Total | NH₃ | NO₃⁻ | TKN |
| | | Concentration | Phosphorus | Nitrogen | Nitrogen | Nitrogen | (mg |
| Substance | Supplier | (mg L⁻¹) | (mg L⁻¹) | (mg L⁻¹) | (mg L⁻¹) | (mg L⁻¹) | L⁻¹) |
| Beef | MP | 1.8 | | 0.216 | | | 0.216 |
| extract | Biomedicals | | | | | | |
| Peptone | BD | 2.7 | | 0.405 | | | 0.405 |
| Humic | Alfa Aesar | 4.25 | | 0.262 | | | 0.262 |
| acid | | | | | | | |
| Tannic | Acros | 4.18 | | | | | |
| acid | Organics | | | | | | |
| SLS | Tokyo | 2.4 | | | | | |
| | Chem. Ind. | | | | | | |
| Gum | Acros | 4.7 | | 0.031 | | | 0.31 |
| arabic | Organics | | | | | | |
| (NH₄)₂SO₄ | Sigma | 2.5 | | 0.530 | 0.530 | | 0.530 |
| K₂HPO₄ | Sigma- | 10 | 1.78 | | | | |
| | Aldrich | | | | | | |
| NaNO₃ | Sigma- | 36.5 | | 9.876 | | 9.786 | |
| | Aldrich | | | | | | |
| MgSO₄ | Sigma | 0.71 | | | | | |

| | | | | | |
|---|---|---|---|---|---|
| Total Concentration (mg L⁻¹) | 1.78 | 11.32 | 0.53 | 9.88 | 1.44 |

“TKN” denotes Total Kjeldahl Nitrogen.
“SLS” denotes sodium lignin sulfonate.

To isolate unicellular microalgae, RAB samples and/or flask cultures were homogenized by vortex mixing or stirring and serially diluted 1:10 into GWT-SE medium to a final dilution of 1:1,000. 100 μL of the dilutions were plated onto solid BG-11, BBM, and GWT-SE media and grown at 100-250 μE m-2 s-1 under white LED panel lights. Potential microalgal colonies (green or brown colonies) were transferred to fresh solid medium and maintained by restreaking approximately every 3 to 8 weeks. Eukaryotic microalgal isolates were axenized by successive streaking on solid medium adjusted to 100 μg mL-1 to 1 mg mL-1 sodium ampicillin (Gold Biotechnology) and 1 μg mL-1 to 5 μg mL-1 carbendazim (Aldrich).

Diatom strains were isolated using the same technique with solid media containing 5 g L-1 silicon dioxide (Sigma-Aldrich). Filamentous microalgae were isolated by sample filtration. A small amount of each biofilm sample (50-450 μL or a 2-3 mm piece) was transferred to a sterile 40-70 μm nylon mesh strainer (Fisher Scientific) over a vacuumed flask. The biofilm was rinsed with approximately 100 mL sterile 1×GWT-SE medium dispensed from a vented wash bottle. Strainers were immersed in a sterile dish containing liquid 1×GWT-SE medium, and 50 μL of biomass remaining on the strainer was spread on solid BG-11 medium and grown at 50-145 μE m-2 s-1 under white LED panel lights. Filamentous green or brown colonies were transferred to fresh solid medium and maintained as described above.

### 2.2 Morphological and Phylogenetic Characterization of Microalgal Isolates

All microalgal isolates were imaged and cataloged by cell morphology via brightfield microscopy as described in Section 2.1. One isolate per unique morphology (including cell size and shape, intracellular vesicles, appendages, chlorophyll distribution, etc.) from each RAB sample was maintained for phylogenetic identification, polyP screening, and cryopreservation.

Microalgae were phylogenetically assigned via 16S/18S rDNA PCR and Sanger sequencing. Colonies of microalgae were transferred from solid medium maintenance cultures to 0.2 mL PCR tubes with 50 μL of sterile lysis buffer containing 20 mM Tris (Biorad; pH 8.0) and 0.1% (v/v) Triton X-100 (Sigma-Aldrich). Tubes were vortex mixed briefly and lysed in a SimpliAmp thermal cycler (Life Technologies) at 60° C. for 6 min and 80° C. for 4 min. Cell-free lysates were also prepared as PCR and sequencing controls. Lysates were stored at −20° C. until PCR amplification.

Lysates were thawed and cell debris was pelleted by centrifugation for 1 min on a LabMini 6M Mini Centrifuge (Southwest Science). 1 μL of lysate supernatant was transferred to a 0.2 mL PCR tube containing the following reaction mixture: 12.5 μL Q5 Hot Start High Fidelity 2×Master Mix (New England Biolabs), 1.25 μL each of 10 μM forward and reverse primers (Table S3; IDT) dissolved in 10 mM Tris (pH 8.0), and 9 μL nuclease-free water (Cytiva Life Sciences). All reactions were cycled on a ProFlex thermal cycler (Life Technologies) as follows: 98° C. denaturation for 30 sec; 30 cycles of 98° C. denaturation for 10 sec, annealing at varying temperatures for 30 sec, and 72° C. elongation for varying times (Table 3); 72° C. elongation for 2 min; 4° C. hold. The annealing temperature for primers D512for/D978rev was determined experimentally for use with both diatom and green alga isolates. Annealing temperatures for all other primer sets were determined with the New England Biolabs Tm Calculator (v1.13.1). PCR amplicons were stored at 4° C. and assessed for purity and length by gel electrophoresis in a 1% w/v agarose gel stained with SYBR Safe (Invitrogen). Gels were imaged on a Fluorchem Q imager with Fluorchem Q software (Cell Biosciences).

TABLE 3
Primers used in this study.

| | Target region | | Annealing |
|---|---|---|---|
| | & expected | | temperature/ |
| Primer | amplicon | Sequence | extension |
| name | length | (5′→3′) | time |
| CYA359F* | 16S rDNA V3-V4 | GGGGAATYTTCCGCAATGGG | 66° C./20 sec |
| CYA781R(a) | region | GACTACTGGGGTATCTAATCCCATT | |
| CYA781R(b) | 446 bp | GACTACAGGGGTATCTAATCCCTTT | |
| D512for* | 18S rDNA V4 | ATTCCAGCTCCAATAGCG | 51° C./20 sec |
| D978rev | region | GACTACGATGGTATCTAATC | |
| | 466 bp | | |
| DIV4for* | 18S rDNA V4 | GCGGTAATTCCAGCTCCAATAG | 60° C./20 sec |
| | region | | |
| DIV4rev3 | 329 bp | CTCTGACAATGGAATACGAATA | 72° C./60 sec |
| ss5 | 18S rDNA gene | GGTGATCCTGCCAGTAGTCATATGCTTG | |
| ss3 | 1823 bp | GATCCTTCCGCAGGTTCACCTACGGAAACC | |
| mod-ss5* | — | CCTGCCAGTAGTCATATGCTTG | — |
| mod-ss3* | | GGTTCACCTACGGAAACC | |

*Primers used to prime sequencing reactions.
Mod-ss5 and mod-ss3 are trimmed ss5/ss3 primers modified for Sanger sequencing priming and were not used for DNA amplification.

Microalgal isolates were identified by colony PCR and amplicon sequencing of the 18S/16S SSU rRNA genes using four previously published primer sets (Table 3). The CYA359F/CYA781R primer set was sufficient for the identification of all prokaryotic isolates. In contrast, multiple primer sets were needed for the identification of all eukaryotic isolates. The D512for/D978rev primer set was used initially for all eukaryotic isolates. This primer set efficiently amplified DNA from diatoms, and lowering the annealing temperature used with this primer set was sufficient to improve amplification in green algae (not shown). However, the hypervariable V3-V4 region amplified by D512for/D978rev was found to offer poor resolution in identifying many isolated green algae. Additionally, the primer set was found to be non-specific and amplified DNA from eukaryotic contaminants. Several isolates from RAB samples collected in Chicago, Illinois in February 2021 were inadvertently found to have been co-isolated with the common bacterivorous amoeba Vermamoeba vermiformis. The ss5/ss3 primer set was also non-specific but amplified the entirety of the 18S gene, offering sufficient sequence coverage to identify green algae that could not be identified by the V3-V4 region alone, given that the culture did not possess eukaryotic contaminants. However, the ss3/ss5 primer set did not amplify DNA from any isolated diatom strain. DIV4for/DIV4rev3 was adapted from the D512for/D978rev primer set and similarly amplified the V3-V4 region but was not susceptible to contaminating eukaryotic DNA. Thus, this primer set was used to sequence and identify isolates for which eukaryotic contaminants were present.

Primer extension sequencing was performed by Azenta Life Sciences (South Plainfield, New Jersey) with Applied Biosystems BigDye v3.1. The sequencing reactions were carried out on an Applied Biosystem 3730xl DNA Analyzer. Sequencing was performed unidirectionally for most isolates (Table 3). Several green algae isolates were sequenced bidirectionally with the mod-ss5/mod-ss3 primer set and unidirectionally with D512for to capture the full 18S rDNA gene. To obtain full 18S rDNA sequences, reverse complement sequences were obtained with the Sequence Manipulation Suite and the resulting unidirectional sequences were merged with EMBOSS. All 16S/18S rDNA sequences were deposited to GenBank under accession numbers OP143966 to OP144056 (eukaryotes) and OP142377 to OP142385 (prokaryotes). A top phylogenetic match was determined for each microalgal isolate based on homology with available 16S/18S rDNA sequences in GenBank using NCBI BLASTn. The tentative phylogenetic assignment for each strain was supported by microscopy.

### 2.3 Phosphorus Content Screening of Isolated Microalgae

To screen the polyP content of isolated microalgae, isolates were cultivated in suspended flask cultures, and polyP was extracted and measured using a fluorometric assay. Isolates were cultivated by transferring microalga colonies to 35 mL liquid medium in 125 mL non-baffled borosilicate Erlenmeyer flasks with aluminum foil caps. BBM amended with different nutrients was used for cultivating the various microalgae isolates (Table 3). Each strain was cultivated for polyP measurement as a single biological replicate. Unicellular microalgae were grown on an Orbital Genie SI-1700 shaker at 100 rpm illuminated at 50-85 μE m⁻² s⁻¹ by white light LED panels. Filamentous microalgae were cultivated under the same conditions without shaking. PolyP content has been shown to fluctuate, often peaking early in the growth curve. As such, cultures were grown to a low cell density to capture the upper range of polyP accumulation, with unicellular cyanobacteria and green algae harvested at a low optical density at 730 nm wavelength (OD730) as measured with a Biochrom WPA Biowave II. Unicellular cyanobacteria and green algae cultures were measured for OD730 every 1-2 days and those exceeding a previously determined minimum detection threshold of the fluorometric polyP assay of OD730 0.5 (data not shown) were harvested. Diatoms and filamentous microalgae, for which even subsampling and accurate OD730 measurements were not possible due to cell aggregation in liquid culture, were grown to a visually determined low cell density over one to three weeks.

Once grown, screening cultures were harvested for polyP and CDW measurements. Cultures were placed on ice, and cells were harvested for polyP measurements by transferring 1 mL culture to a microcentrifuge tube and adjusting to a final concentration of 0.001% (v/v) Triton X-100 to improve harvesting yield. For non-homogenous cultures, the entire culture was adjusted to 0.001% (v/v) Triton X-100 and homogenized by pipetting and vortex mixing the culture before transferring the culture to microcentrifuge tubes. Tubes were centrifuged at 16,000×g for 5 min and the supernatant was removed. Cell pellets were washed in ultrapure water containing the same concentration of Triton X-100 and recentrifuged as before. The supernatant was removed, and cell pellets were stored at −80° C. until polyP measurement. A RAB biofilm (microalgae consortium) from a pilot-scale RAB system treating municipal secondary effluent in Slater, Iowa was also diluted 1:100 in ultrapure water and harvested as described above for polyP measurement.

CDWs were determined by filtering two 10 mL aliquots of each culture through pre-rinsed, dried, and weighed 0.7 μm hydrophilic glass fiber filters (Millipore Sigma) over a vacuumed Buchner funnel. Filters were dried for 48 hours at 60° C. and reweighed. The CDW value of the microalgae consortium was determined by centrifuging triplicate 20 mL biofilm samples at 5,000×g for 15 min, removing the supernatant, freeze-drying the biofilm in a Harvest Right HRFD-PMED-WH freeze-dryer, and weighing the dried biomass.

Phosphorus accumulation by each isolate and the microalgae consortium was quantified by extracting and measuring polyP. To extract polyP, 300 μg (CDW) of harvested cell material was suspended in 600 μL of 10 mM HEPES buffer (pH 6.8) and transferred to 2 mL screwcap tubes containing 600 u L of 0.1 mm silica/zirconia beads (BioSpec). Samples were vortex mixed and boiled on a dry heat block for 5 min at 100° C. Samples were then cooled on ice, and cells were lysed in an Eppendorf Tissue Lyzer II for 5 min at 30 Hz. Cell debris and beads were removed by centrifuging twice at 16,000×g for 3 min, transferring lysate supernatant to fresh tubes each time. The recovered lysate containing the extracted polyP was treated with enzymes to remove biomolecules known to interfere with fluorometric polyP quantification. 300 μL of supernatant was treated consecutively at 37° C. with 5 μL Ambion RNase Cocktail for 10 min, 5 μL Invitrogen TURBO Dnase for 10 min, and 10 μL Roche Proteinase K for 30 min. Blank solutions were prepared with the same protocol but without cells. PolyP concentration in the enzyme-treated lysate was quantified using the ProFoldin MicroMolar PolyP Assay Kit (Cat. No. PPD1000) according to the manufacturer's protocol. Fluorescence measurements were taken in Andwin Scientific 96-well clear-bottom black microtiter plates (Cat. No. 655096) with a Tecan Infinite M200 Pro plate reader.

### 2.4 Cryopreservation and Culture Collection Submission of Isolated Microalgae

Microalgae isolates were cryopreserved with protocols adapted from Elliott et al. (2012) and the Culture Collection of Algae at the University of Texas at Austin (UTEX). Isolates were cryopreserved as either liquid cultures or agar slant cultures. Briefly, liquid-cultured isolates were cultured in 20 mL BBM in 50 mL non-baffled borosilicate Erlenmeyer flasks with aluminum foil caps. Upon reaching OD730 0.5-1.2, 1.9 mL aliquots of the cultures were transferred to 2 mL cryotubes (Corning), adjusted to 5% (v/v) DMSO (Sigma-Aldrich), gently mixed, and cooled to-80° C. in Mr. Frosty (Nalgene) freezing containers before being transferred to a liquid nitrogen cryopreservation tank (−196° C.) for long-term storage. Slant-cultured isolates were cultured as streaks on 1-mL BBM agar slants (Table S1) prepared in 2 mL cryotubes. Slant cultures were gently overlaid with 800 μL of liquid BBM containing 5% (v/v) DMSO and cryopreserved as described above. Diatoms were cryopreserved as described above, but with a DMSO concentration of 12% (v/v).

In addition to cryopreservation, a selection of P-hyperaccumulating and diverse microalgae isolated in this study were submitted to UTEX (https://utex.org/) and are publicly available. Submitted strains can be searched on the UTEX website using the identifiers assigned in this study (e.g., TCF-8d) or the keywords “wastewater remediation” or “polyphosphate accumulation”.

### 2.5 Bench-Scale RAB Colonization and Phosphorus Removal Testing

Four bench-scale RAB systems, each with a 4 L reservoir and a cotton belt with 0.1 m² surface area were used to assess strain performance in a RAB system in comparison to a naturally occurring consortium. The experiment was conducted in a non-sterile greenhouse in Boone, Iowa in March 2022 at 20° C. and supplemented with constant artificial illumination by custom-made white LED panel lights (Reliance Laboratories) at 200 μEm⁻² s⁻¹. Chlamydomonas pulvinata TCF-48g and Chlorellaceae sp. TCF-17g were selected for testing due to their high P content and fast growth, respectively. Strains possessing higher levels of polyP (i.e., diatoms) were not included, as they had not yet been screened for polyP at the time of RAB testing. The same RAB microalgae consortium included as a community baseline in the polyP screen was also included as a community baseline in this test.

To prepare seed cultures, 100 mL of a low-density BBM culture of each strain or a 1:100 dilution of the RAB microalgae consortium into ultrapure water was inoculated into triplicate Erlenmeyer flasks containing 900 mL BBM. Seed cultures were grown for four to seven days in a Sanyo MCO-17AI CO2 Incubator on a Velp Scientifica magnetic multi-stirrer at 100 rpm, 32° C., and 2% CO2. Each of the four bench-scale RAB system reservoirs was filled with 2 L of seed culture (Chlamydomonas pulvinata TCF-48g, Chlorellaceae sp. TCF-17g, a 1:1 mixture of both strain seed cultures, or the natural consortium) and 2 L of fresh BBM. BBM was chosen for this preliminary test in consistency with the conditions used for polyphosphate screening.

The RAB systems were operated at a constant speed of 6 rpm with a velocity of 6 cm s-1 for three weeks with a semi-continuous flow of BBM. Approximately 500 mL of ultrapure water was added to each RAB reservoir daily to accommodate for evaporation and maintain a total solution volume of 4 L. Every seven days, the biomass on the belt was observed with an Olympus CX31 microscope to verify belt colonization by the respective strain(s) and harvested by scraping into 50 mL conical tubes. Harvested biomass was centrifuged at 5,000×g for 15 min, the supernatant was removed, and the biomass was freeze-dried as in Section 2.3. Freeze-dried biomass samples were stored at −20° C.

After the first belt harvest at 7 days, 2 L of RAB reservoir solution was drained from each RAB system and replenished with 1 L of fresh seed culture and 1 L of fresh BBM. After the second harvest at 14 days, 2 L of solution was drained from each RAB reservoir and replenished with 2 L of fresh BBM. Total P fed into each RAB system throughout the experiment (as fresh media or seed culture) was 424 mg P in 8 L, calculated using the media recipe. The freeze-dried harvested biomass samples were weighed to determine biomass productivity and sent to Midwest Laboratories, Inc. (Omaha, Nebraska) for total P content analysis using inductively coupled plasma mass spectrometry (ICP-MS). Biomass productivity measurements were collected for each of the three harvests from each RAB belt. ICP-MS P measurements were collected for biomass from the first harvest and for pooled biomass samples from both the second and third harvest from each RAB belt.

### 3. Results

### 3.1 Biodiversity in the Wastewater Treatment Biofilms

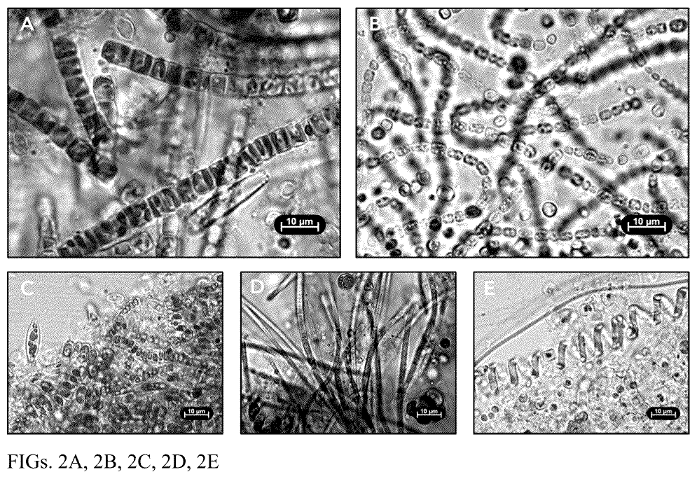

RAB samples were imaged upon arrival and possessed a large diversity of microalgae, including cyanobacteria, diatoms, and green algae (FIG. 2). RAB systems notably also supported diverse organisms of higher trophic levels, including bacteria; filamentous fungi; protozoa including amoebae, euglenids, and ciliates; and animals including nematodes, rotifers, and tardigrades. RAB community compositions varied drastically across RAB system locations and sampling time points. This suggests that RAB microbial communities are dynamic and may change with fluctuations such as seasonal shifts in light and temperature or influent composition.

### 3.2 Strain Isolation and Morphological Characterization

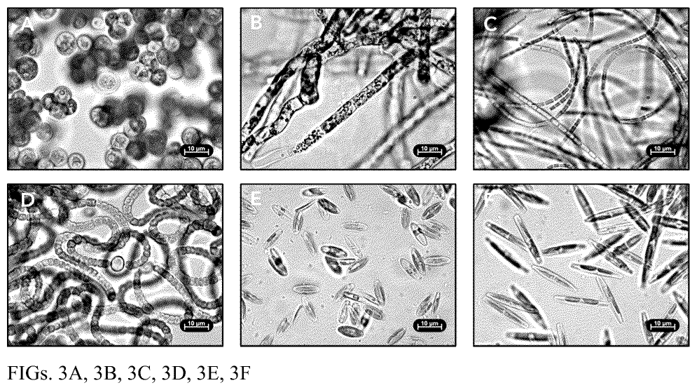

Approximately 770 microalgal colonies were obtained in total from the samples collected from eight active RAB systems across three time points. Microscopic imaging of each colony revealed a diverse range of green algae (FIG. 3AB), cyanobacteria (FIG. 3CD), and diatoms (FIG. 3EF) among the isolates. Microscopy images were used to identify morphologically unique isolates for retention in the culture collection. One isolate per distinct morphology from each sample was considered unique and retained in the culture collection (i.e., multiple isolates of the same morphology were retained if they originated from different RAB samples). A total of 101 strains were retained in the culture collection. Targeted isolation techniques, including silicon enrichment and RAB sample filtration, were successful and resulted in the isolation of nine diatoms and ten filamentous microalgae, respectively.

### Genetic Identification of Isolated Microalgae

Microalgae retained in the culture collection were identified by 16S/18S rDNA sequencing and microscopy. A representative microscopy image of each strain, as well as the RAB microalgae consortium used in this study, is available in Supplemental File S1. It should be noted that many isolated strains display varying morphology between cells attributable to the species' life cycle. High-quality rDNA sequences were obtained for 100 of the 101 isolated strains. Of these sequenced strains, 97 strains were assigned tentative phylogenetic matches at the genus or species level.

Of the 101 microalgae isolated, 82 were green algae, with the vast majority assigned to the Scenedesmaceae (51) and Chlorellaceae (21) families. Green algae strains were assigned as Chlamydomonas pulvinata, Caespitella pascheri, and Chlorolobion spp. were also isolated. Additionally, nine diatom strains were isolated and assigned as Craticula molestiformis, Eolimna minima, Nitzschia palea, and Nitzschia sp. Ten cyanobacteria strains were isolated and assigned as Synechocystis sp., Leptolyngbya spp., Nodosilinea spp., Nostoc edaphicum, and Pseudanabaena sp.

### 3.4 Polyphosphate Content Screening of Isolated Microalgae

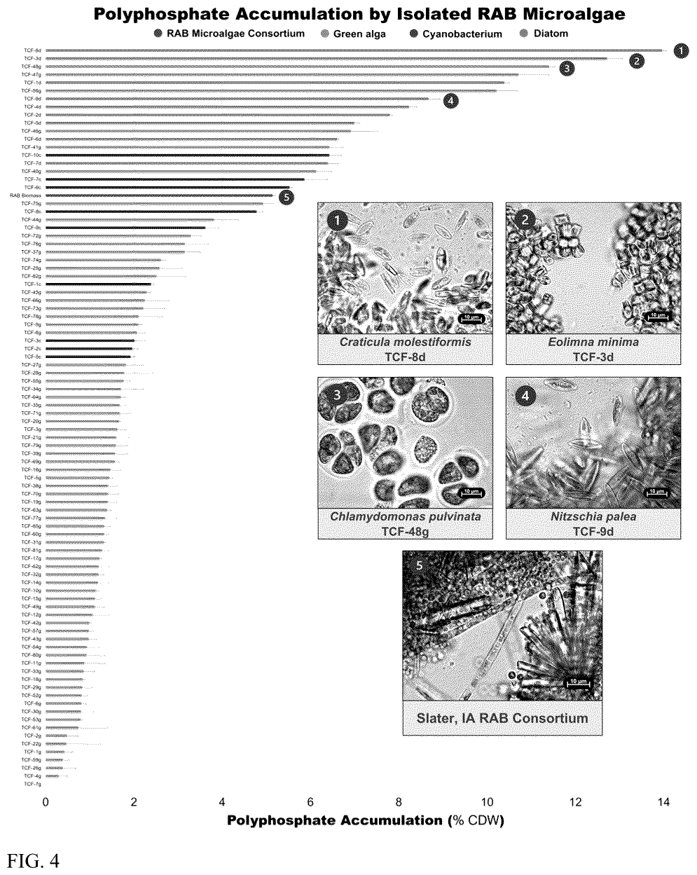

Microalgae isolates were screened for P accumulation by extracting and measuring polyP. PolyP measurements were obtained for 91 of the 101 strains retained in the culture collection, including all strains that could be successfully cultivated in liquid BBM media (FIG. 4). A RAB microalgae consortium sampled from a RAB system in Slater, Iowa was also screened to serve as a baseline representing P accumulation in existing RAB systems. The RAB microalgae consortium accumulated 5.1% polyP by CDW. In comparison, seven isolates accumulated at least 50% more polyP, including three isolates of Chlamydomonas pulvinata, two isolates of Craticula molestiformis, and one isolate each of Eolimna minima and Nitzschia palea. The top P-hyperaccumulating strain, Craticula molestiformis TCF-8d, accumulated 14.0% polyP by CDW.

Notably, all green algae were found to be below the RAB microalgae baseline, except for multiple isolates of Chlamydomonas pulvinata. All nine diatom strains and three filamentous cyanobacteria strains accumulated more polyP than the RAB microalgae consortium.

3.5 Recolonization and Performance of Chlamydomonas pulvinata TCF-48g in Bench-Scale RAB Systems

As an initial test to show that isolated P-hyperaccumulating microalgae can recolonize the RAB system and improve P removal, one P-hyperaccumulating green alga strain, Chlamydomonas pulvinata TCF-48g (FIG. 4 panel 3), was tested for recolonization, biomass productivity, and total P accumulation in a bench-scale RAB system containing BBM medium. At the time of testing, C. pulvinata TCF-48g was the highest P-accumulating strain identified. A RAB microalgae consortium from a Slater, Iowa RAB system (FIG. 4 panel 5) and a fast-growing, low P-accumulating isolate Chlorellaceae TCF-17g were also tested in parallel for comparison.

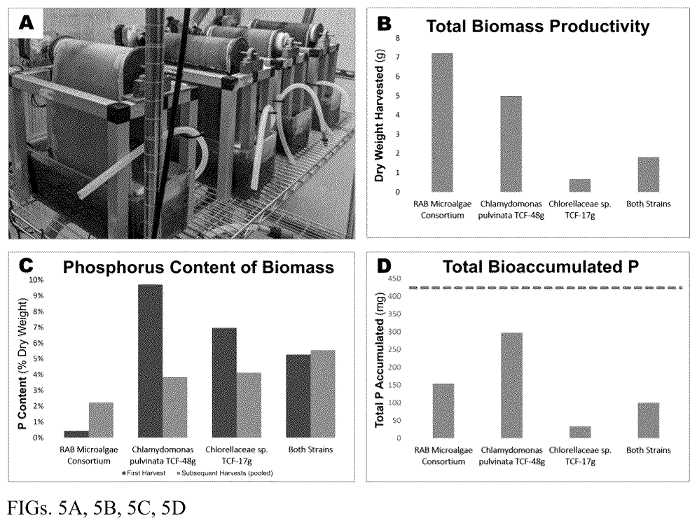

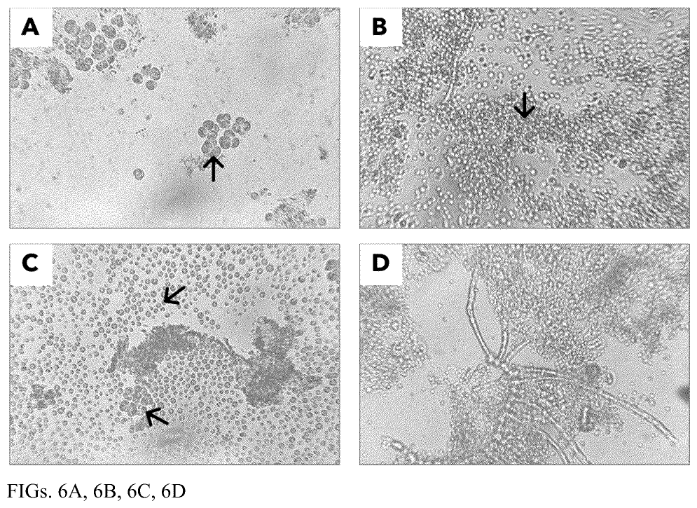

All four inocula successfully colonized the RAB belts (FIG. 5A, FIG. 6). However, stark differences in productivity were observed between the inocula, with the RAB microalgae consortium producing the greatest amount (7.2 g) of total dry biomass throughout the experiment (FIG. 5B). The P-hyperaccumulating strain C. pulvinata TCF-48g was 31% less productive, producing 5 g of dried biomass.

Of the harvested biomass from the RAB systems, the RAB microalgae consortium possessed the lowest P content, ranging between 0.43% CDW total P at the initial harvest and increasing to 2.25% P in subsequent harvests (FIG. 5C). The isolated P-hyperaccumulating C. pulvinata TCF-48g possessed approximately 22-fold more P than the RAB microalgae consortium in the initial harvest, containing 9.7% P. However, this value decreased to 3.9% P in subsequent harvests.

Taking together the productivity and P content data, the harvested biomass from C. pulvinata TCF-48g contained 297 mg P out of the 424 mg P added to each RAB reservoir throughout the experiment. This equates to a P removal rate of 70% under the conditions tested, which is two-fold greater than that of the RAB microalgae consortium, which removed 36.4% P (FIG. 5D). It should be noted that any additional bioaccumulated P in microalgae biomass remaining on the belt after scraping or in the reservoir was not measured.

### 4. Discussion

4.1 Diversity of Algal Strains Isolated from RAB Biofilm

RAB systems are an emerging algal technology for wastewater treatment, but to date, the microalgal composition has not been characterized. We found that the microbial community composition of RAB systems varies drastically by system location, influent composition, and season of sampling. This work isolated 101 microalgal strains from these RAB system microbial communities. Further, the targeted isolation of diverse microalgae from these samples was successful. Diatoms were isolated by supplementing media with silicon dioxide, and filamentous microalgae were isolated by filtering the RAB samples before plating. While these techniques proved useful in isolating microalgae based on morphology and nutritional requirements, other media compositions and isolation techniques may further improve the diversity of strains isolated for future microalgal bioprospecting efforts.

### 4.2 Diversity and Dynamics of Algal Polyphosphate Accumulation

This work identified significant variation in polyP accumulation among microalgal strains, ranging from 0% to 14% CDW. This equates to 0% to 5.4% P attributable to polyP (with elemental P accounting for approximately 40% of polyP by weight). It should be noted that cells typically possess an additional 1% elemental P attributable to other phosphorous biomolecules (e.g., nucleic acids, phospholipids) not measured here. As a reference, the RAB microalgae consortium possessed 5.1% CDW polyP, or approximately 3% total P including other P biomolecules, in the polyP screen and between 0.43% to 2.25% CDW total P in the bench-scale RAB testing. This is consistent with previous research demonstrating that naturally occurring microalgae in waste stabilization ponds possess between 0.21% to 3.85% P by CDW.

Notably, our results suggest that P accumulation varies across different phylogenetic groups. Diatom strains generally accumulated higher levels of polyP than most green algae strains (FIG. 4). This is a significant finding considering previous microalgal bioprospecting work for resource recovery has focused heavily on green algae. Diatoms are known to accumulate high levels of both phosphorus and nitrogen as a means to survive under unfavorable. Previously, it had been discovered that even under low P, a condition known to inhibit polyP synthesis in most microalgae, the diatom Thalassiosira pseudonana increased polyP synthesis. Further research into P metabolism in diatoms is needed to fully understand and exploit these unusual polyP dynamics. The screening data presented in this study indicate that diatoms may possess an enormous amount of unexplored potential as tools to achieve a circular nutrient economy. These data also demonstrate that diverse strain isolation in bioprospecting work is key to identifying valuable strains for resource recovery from wastewater.

In this work, the polyP content of most strains was screened within a single culture condition and at a single time point within their growth. However, it has been reported that polyP accumulation can fluctuate in microalgae depending on nutrient availability and growth phase. For example, others have found that two out of three tested green algae strains accumulated high levels of polyP after two days of growth in municipal wastewater, but the polyP content decreased rapidly thereafter. Similarly, others have observed that the cyanobacterium Synechocystis sp. PCC 6803 accumulated polyP within minutes of surplus phosphate exposure, but that polyP levels decreased rapidly and depleted within 2 days. Thus, the single time point used for strain screening may have missed the peak P accumulation capacity in some strains, precluding the discovery of other algal strains capable of P hyperaccumulation. Measuring polyP under varying conditions and at multiple time points during strain growth could elucidate the full capacity and dynamics of P accumulation by the microalgae strains isolated in this study.

### 4.3 Application of the Algal Strain Collection

The P-hyperaccumulating microalgae isolated in this study have the potential to boost the P removal of RAB systems. This work included an initial test of recolonization and strain performance of two algal strains in bench-scale RAB systems containing BBM medium. The tested strain C. pulvinata TCF-48g was able to recolonize the RAB biofilm and outperform a RAB microalgae consortium in P removal. Further, the P-rich biomass produced from this strain possessed up to 9.7% P, higher than the 5-7% P typically found in biosolids produced by enhanced biological phosphorus recovery, a competing biological P-removal technology. This observation is consistent with the high polyP content of this strain grown in a flask during the polyP content screen (FIG. 4). However, the high level of total P measured in the bench-scale RAB testing was observed only during the first harvest of algae biomass from the RAB system and decreased to 3.9% P in subsequent harvests. The reduction in the P content of this strain in subsequent harvests may have been due to the exhaustion of available phosphate in the medium. 70% of the P added to the RAB system was recovered in C. pulvinata TCF-48g biomass scraped from the belt (FIG. 5D). However, residual algae biomass remaining on the RAB belt after scraping and in the RAB reservoir also bioaccumulated P that was not measured. Further, precipitation of phosphate may have occurred in the system, though this likely would have been negligible due to the high proportion of P recovered in algae biomass. Therefore, it is conceivable that the available P in the medium had been depleted at the time of the second and third harvests. The P accumulation observed in this strain during the initial harvest may thus represent the true upper limit of P accumulation by this strain in conditions where phosphate is not limited, such as in the conditions of commercial-scale RAB systems, in which wastewater is supplied continuously.

In an embodiment, methods and systems disclosed herein demonstrate a 100% improvement in the phosphorus removal rate (e.g., from 36% removal baseline to 72%) while retaining algal biomass productivity during tertiary wastewater treatment at pilot scale.

While these preliminary test results are promising, whether other P-hyperaccumulating microalgae isolated in this study can easily recolonize RAB systems and similarly outperform RAB microalgae consortia remains to be studied. Furthermore, the performance of isolated P-hyperaccumulating microalgae in RAB systems treating wastewater (rather than synthetic medium) remains to be tested. The inoculation of polycultures of multiple P-hyperaccumulating strains with varying ecological niches may be a useful approach to improve inoculum colonization and resilience in RAB systems, further highlighting the benefit of diverse strain isolations in bioprospecting research.

Additionally, the P-hyperaccumulating strains isolated in this study may be valuable for other waste remediation applications, such as metal removal and recovery from industrial (e.g., mining) and municipal wastewater. PolyP is a highly negatively charged polymer known to chelate and accumulate cationic metals in microalgae. Uranium, lead, copper, silver (Ballan-Dufrançais et al., 1991), cadmium, and other metals have been shown to concentrate in microalgal polyP bodies. As such, P-hyperaccumulating microalgae may be useful in both recovering valuable metals and removing harmful metals from wastewater streams. RAB systems have previously been found to remove metals from wastewater, including chromium, manganese, copper, and other metals from municipal sludge thickening supernatant and high levels of nickel from synthetic media, although the relationship between biofilm polyP levels and metal accumulation has not been studied. RAB systems and other algal waste remediation technologies may benefit from the use of P-hyperaccumulating microalgae with greater capacity to take up and store metals from wastewater.

While these strains were isolated to improve P removal by RAB systems, the P-hyperaccumulating microalgae isolated in this study could potentially be used to improve P removal in other microalgal wastewater treatment systems (e.g., waste stabilization ponds or photobioreactors). Additionally, these microalgae isolates may also possess other valuable phenotypes for downstream applications of wastewater-grown algal biomass, such as high lipid content for biofuel production. Future screening efforts may thus identify other valuable microalgae strains isolated in this study.

Overall, this work demonstrates the potential of bioprospecting as an effective approach to improve resource recovery from wastewater by microalgae. Of the 101 microalgae strains isolated in this study from RAB systems, multiple species of diatoms and one species of green alga were found to accumulate high levels of polyP. These isolates may serve as valuable tools to remove and recover P and other resources from wastewater.

We claim:

1. A method for enriching a microbial population capable of sequestering phosphorus and nitrogen comprising the addition of silicon rich materials to a solution wherein the microbial population is contacted with the solution.

2. The method of claim 1 wherein the silicon rich material is sand.

3. The method of claim 1 wherein the microbial population is cyclically exposed to the solution and further exposed to atmosphere.

4. The method of claim 3 wherein the atmosphere comprises carbon dioxide.

5. The method of claim 1 wherein the microbial population comprises Chlamydomonas pulvinata TCF-48g.

6. The method of claim 1 wherein the microbial population comprises diatoms Eolimna minima TCF-3d.

7. The method of claim 1 wherein the microbial population comprises Craticula molestiformis TCF-8d.

8. The method of claim 1 wherein the weight percent of phosphorus of the dry weight of the microbial population biomass is from about 11 percent to about 14 percent.

9. A method for the removal of phosphorus from a solution comprising enriching a microbial population capable of sequestering phosphorus and nitrogen comprising the addition of silicon rich materials to a solution wherein the microbial population is contacted with the solution and further comprising isolating the microbial population and isolating phosphorus from the microbial population.

10. The method of claim 9 wherein the silicon rich material is sand.

11. The method of claim 9 wherein the microbial population is cyclically exposed to the solution and further exposed to atmosphere.

12. The method of claim 11 wherein the atmosphere comprises carbon dioxide.

13. The method of claim 9 wherein the microbial population comprises Chlamydomonas pulvinata TCF-48g.

14. The method of claim 9 wherein the microbial population comprises diatoms Eolimna minima TCF-3d.

15. The method of claim 9 wherein the microbial population comprises Craticula molestiformis TCF-8d.

16. The method of claim 9 wherein the weight percent of phosphorus of the dry weight of the microbial population biomass is from about 11 percent to about 14 percent.
